(19) United States
(12) Patent Application Publication
(10) Pub. No.: US 2008/0206795 A1
(43) Pub. Date: Aug. 28, 2008

(54) Novel Supports, in Particular for Immunodetection of Molecules of Interest

(76) Inventors: Ernst Heinen, Villers St-Simeon (BE); Gilbert Legeay, Saint Saturnin (FR); Fabienne Poncin-Epaillard, Le Mans (FR); Willy Zorzi, Seraing (BE); Benaissa Elmoualij, Liege (BE); Veronique Legeais, Le Mans (FR); Eric Rouault, Sarge Les Le Mans (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/910,554

(22) PCT Filed: Apr. 4, 2006
(86) PCT No.: PCT/BE2006/000030

§ 371 Date: Feb. 22, 2008

(30) Foreign Application Priority Data
Apr. 4, 2005 (FR) 05 03322

(51) Int. Cl. G01N 33/53; B05D 3/00; C12M 1/24; C12P 19/34

(52) U.S. Cl. 435/7.92; 427/532; 435/304.1; 435/294.1; 435/91.2

(57) ABSTRACT

The invention concerns supports or receptacles for solubilized or suspended biological entities whereof the walls are made of plastics surface-treated by an electromagnetic plasma followed by a polymer deposition. It can be in the form of tubes or multiple-well plates. It provides among other things the possibility of storing, transferring and performing reactions in the context of microassays of biological entities, more particularly the prion protein, with enhanced sensitivity.

The present invention relates to novel types of supports designed mainly for the detection and assaying of molecules of interest, in particular those present at very low concentrations. These supports can also allow the transfer, storage and handling of molecules of interest.

There are many intended fields of application. The present invention relates, however, more particularly to the immunodetection, the selective capture, the handling and the storage of molecules of interest. These supports may therefore be used in the agrofoods and animal and human health industrial sectors, and more particularly in medical or veterinary test laboratories. By way of indication, in worldwide terms, the market for in vitro diagnosis represents 24 billion euros, including 7.4 million just for Europe (900 million for instruments and 6.5 billion for reagents). The detection and the assaying of specific antigens are therefore of considerable economic importance in the medical diagnosis, environmental and agrofoods field; in particular, in the veterinary field for the detection of the prion protein in cattle (bovine spongiform encephalopathy), members of the sheep family (sheep scrapie) and members of the goat family; and in the field of human medicine for the detection of the prion protein and of biomarkers for neurodegenerative diseases.

At the current time, the need to improve the sensitivity of in vitro diagnostic tests is constantly felt, in particular in order to be able to diagnose diseases at an early stage, prior to the appearance of the first clinical symptoms. The development of such diagnostic kits therefore requires the integration of three essential criteria, namely:
the preparation of the samples, and more particularly the protocol for extracting the protein from the matrix;
the supports (tubes, pipettes, tips,), which should be treated in such a way as to in particular improve the binding of the molecule of interest (antibody or antigen);
the method of detection, which should be ultra-sensitive (for example: quantitative immuno-PCR).

The invention is aimed at improving the preparation of the sample according to the type of antigen that it is desired to detect, more particularly as regards the support.

The use of increasingly sensitive detection technologies brings about an exacerbation of the role of the support, and in particular of its “properties”. In fact, these ultrasensitive technologies are often used to carry out an antigen detection at a concentration very close to the limit of detection. It is therefore essential to be able to optimize the material so as to minimize its interferences and to allow the background noise to be reduced. Most in vitro diagnostic tests, that need immobilization of the molecule of interest to be assayed, require the use of supports that allow the diagnosis to be carried out; but also systems that maximally reduce losses, in particular losses due to adsorption onto the walls, or during storage or sampling of material.

Prion diseases (or transmissible spongiform encephalopathy) are fatal neurodegenerative diseases which affect both humans and animals (bovine and feline spongiform encephalopathy, scrapie in sheep and goats for animals; Creutzfeldt-Jakob disease, Gerstmann-Sträussler-Scheinker disease, fatal familial insomnia or kuru for humans). All prion diseases share the same molecular mechanism, which involves the conversion of a normal cellular prion protein (PrPc) into an infectious form, which is insoluble in nonionic detergents and partially resistant proteases, generally called PrPsc. This infectious protein has a secondary structure particularly rich in β-sheets, which gives it a very hydrophobic nature; it therefore has a tendency to aggregate so as to form water-insoluble fibrilles. In laboratories, what is generally carried out with a recombinant form of the normal prion protein (PrPrec), which is produced from Escherichia coli.

Up until now, no detection kits exist for detecting the infectious prion protein in living humans and animals. In fact, the tests currently used in the food sector for bovine carcass analyses are all based on a post-mortem diagnosis. The latter consist in general of a confirmation by histopathology or immunohistopathology. A series of “rapid” post-mortem tests has emerged over the last few months. They are all based on the use of antibodies: sandwich ELISA (Bio-Rad), direct ELISA (Enfer Scientific) or else Western blotting (Prionics).

Furthermore, over the last few years, a new Creutzfeldt-Jakob variant (vCJD) has appeared. It has been shown that this disease is related to contamination of the individual with the prion protein subsequent to the ingestion of meat infected with mad cow disease. It has also been shown that this disease can be transmitted via blood (by transfusion). Mid-2005, close to 180 individuals were suffering from vCJD in Europe. Out of the 158 detected in England, 148 met the criteria for donating their blood. Since 2004, 3 individuals have already died following a transfusion with blood contaminated by the prion protein.

Given that there are more than 80 million blood bags which are distributed each year worldwide, it therefore becomes obvious that it is necessary to be able to develop a kit for detecting the prion protein which is sufficiently sensitive and sound to enable its detection in blood, in order in particular to be sure that transfused blood bags are healthy and to prevent a new “contaminated blood” affair.

The development of a kit for detecting an infectious protein generally begins with the technology being developed on the recombinant form of this protein, which is noninfectious and therefore easier to handle. In the case of the prion protein, the present invention proposes a detection kit using the ELISA and iPCRq technologies on the recombinant prion protein, PrPrec. It will be understood that, depending on the type of assay to be carried out, the nature of the support will have a considerable influence.

### Sandwich Immunodetection:

In this case, it is necessary to coat a capture antibody onto the surface of the support enabling the diagnostic test to be carried out. According to the type of antibody used, the nature of the support will have to be optimized; since an antibody is a molecule that is soluble in an aqueous medium (serum), it will therefore prefer to bind to a support that is hydrophilic in nature.

### Direct Immunodetection:

In this case, it is necessary to directly coat with the molecule to be assayed. Taking the case of the prion protein as an example, since this protein is highly hydrophobic in the infectious form, it will be preferable to use a support with a hydrophobic nature. This is because, since this protein is repelled by the aqueous solution, it will seek to position itself in a hydrophobic environment and will therefore be greatly attracted by the walls of such a support.

### Storage and Transfer:

Still for the prion protein, since it is highly hydrophobic in the infectious form, it is therefore preferable to store it in receptacles whose wall has a hydrophilic nature. Thus, in this environment, the prion protein will have a greater tendency to be repelled far from the walls and will not tend to go and adhere thereto. There will therefore be less loss of protein of interest.

The material used for the transfer and the handling of this protein will therefore also have to have a hydrophilic nature in order to prevent losses of material on the walls.

For the same antigen, it will therefore be necessary to use various types of receptacles, or supports, according to the use that it is desired to make thereof.

The invention therefore proposes novel types of supports for the detection and assaying of molecules of interest. These supports can also be used for the storage of samples (complex or simplified) and also for the handling of the molecules of interest (sampling systems which reduce sample losses on the walls). These supports will be different in that the wall has under-gone an electromagnetic plasma treatment and polymer deposition which modifies the nature of its surface, according to the use that it is desired to make thereof.

All the supports which have been used in the examples that follow are commercial supports, already optimized by the manufacturers, which have been treated with electromagnetic plasma followed by polymer deposition. In virtually all cases, this treatment further enhanced the performance levels of these supports for carrying out an immunodetection. It should be noted that the invention does not apply only to supports already present on the market, but that other types of supports can be produced and treated according to the invention.

These supports are advantageously intended for carrying out tests for molecular detection and/or assaying by ligand-PCR or immunodetection techniques, such as ELISA, i-PCR or i-PCRq.

### PRIOR ART

The treatment of polymer surfaces with plasma is known. Mention will, for example, be made of the publication “Surface engineering of biomaterials with plasma techniques”, F. PONCIN-EPAILLARD and G. LEGEAY, J. Biomater. Sci. Polymer Edn, Vol. 14, No. 10, p. 1005-1028 (2003).

Moreover, various immunodetection techniques are currently known which use plates (multiwell or not) as support and to the surface of which specific ligand/receptor couples and/or antigens and/or capture anti-bodies have been bound by chemical bonds.

During immunodetection by ELISA, the various molecules of interest are recognized and assayed with a sensitivity threshold which is generally of the order of one nanomolar. The immuno-polymerase chain reaction (i-PCR) has a sensitivity threshold of the order of one femtomolar, by virtue of the fact that the detection of the antigen is carried out subsequent to PCR amplification of a DNA reporter coupled to an antibody specific for the molecule of interest. Immunodetections by quantitative i-PCR (i-PCRq) and by ligand-PCR are introduced in patent document WO 01/31056.

With these very low sensitivity thresholds, the micro-(and nano-) miniaturization of the technologies becomes possible and allows microassaying of prion proteins, and more generally, of proteins. However, with such a sensitivity, it is normal for parasitic phenomena related to the “quality and/or the nature (properties)” of the support surfaces to appear. The miniaturization of tests for detection and/or of assaying, by using samples of ever decreasing volumes and concentrations, leads to an exacerbation of the importance of the surface of the support. This is also true in the field of sample handling, and also that of storage, in which it is preferable for the support to attract the antigen or to repel it, according to the intended applications.

The invention is therefore directed toward controlling the functionality, the uniformity and also the reproducibility of the surfaces of supports, in particular for microassays and for the storage of some molecules.

At the current time, the use of synthetic polymers is very widespread in the manufacture of the various consumables used in the in vitro diagnosis field. The importance of the interaction of the reagents with these supports has in particular stimulated the development of various set ups for improving the binding of antigens to the surface of said supports:
“Plasma treatment of polymeric materials to enhance immobilization of analytes e” (patent No. WO 95/34814).

This invention provides a method for the surface treatment of a polymeric material with a gas plasma (ionized oxygen). This treatment makes it possible to increase the immobilization of the antigen used to perform the diagnosis.
“Method of improving the biocompatibility of solid surfaces” (U.S. Pat. No. 4,973,493).

This invention provides a method for the modification of a solid surface for the purpose of enhancing its biocompatibility. This method uses a biocompatible agent capable of activating covalent bonds with the surface of the solid.
“Immobilized biomolecules and method of making same” (U.S. Pat. Nos 5,034,428 and 4,829,098). This invention provides a method for the immobilization of biomolecules on a support. In said method, the biomolecule itself is grafted onto a solid and hydrophilic polymeric support that has been ionized beforehand. This technique is applicable to the immobilization of a large variety of biomolecules such as enzymes, hormones, lectins, drugs, vitamins, antibodies, etc. These products can be used for therapeutic or diagnostic applications.
“Radio frequency plasma deposited polymers that enhance cell growth” (U.S. Pat. No. 4,919,659). This document discloses a method for increasing the capacity for binding cells in culture. The surface of the material is subjected to a gas plasma discharge which causes the deposition of a polymer on the exposed material. This treatment increases the adsorption, and therefore the attachment, of the growing cells. The preferred gases are acetone oxide, methanol oxide and ethylene oxide.

According to the invention, several types of commercial supports, having the size characteristics desirable for the analyses (dishes, flat supports, multiwell systems, beads, etc.), undergo a surface modification. They can be made of polystyrene (PS), polyester terephthalate (PET), polycarbonate (PC) or else polypropylene (PP). These materials are hydrophobic, i.e. they are difficult to wet, and moreover they cannot be bonded (or made to adhere).

The invention therefore proposes to specifically functionalize these supports, using a clean, reproducible and readily industrializable technology, known as “electromagnetic plasmas”. The latter is carried out by a treatment with gas plasma of varying nature, pressure, power and frequency; followed, in certain cases, and preferably, by a physical treatment which deposits on the surface of the support polymers of medical grade or other organic molecules, polyvinyl-pyrrolidone and celluloses, etc.

These supports have been tested by carrying out immuno-assays by ELISA and iPCRq. The results are applicable to other ultrasensitive techniques such as that of ligand-PCR, which techniques are described in the abovementioned document WO 01/31056. Surprisingly, a clear enhancement in the sensitivity has been observed at the very low concentrations.

The present invention therefore proposes, inter alia, new types of supports (of tube, tip and multiwell plate type) which allow the detection, assaying, handling and storage of molecules of interest.

For the applications aimed at the detection and/or assaying of molecules of interest, these supports according to the invention are “activated” by coating their surface with specific antibodies, antigens, ligands and/or receptors bound via physicochemical bonds.

According to one aspect of the invention, the support is “neutralized” and consequently makes it possible to improve the sensitivity of the assay (mainly at low concentrations), but also to improve the reproducibility of the assay. These supports can in particular be used for the detection and assaying of prion proteins, as described in the examples below.

The invention has been applied more particularly to multiwell supports made of polypropylene (PP), polystyrene (PS) and polycarbonate (PC), but can be applied to other types of supports, such as commercial products, laboratory consumables (tips, tubes, etc.) and medical consumables (catheters, etc), and artificial organs.

According to the invention, it is proposed to treat the biocompatible materials and the supports with an electromagnetic plasma followed by polymer deposition.

The invention will be understood more clearly upon examination of the nonlimiting examples which follow.

### Obtaining the Supports

The “electromagnetic plasma” technology is used. It involves electrical discharges carried out either at ambient pressure (in this case, they are called crown, corona or DBD discharges), or under a partial vacuum (approximately 0.1 to 5 torr, i.e. 1 to 10 mPa). These electrical discharges contain excited atoms of gas (N₂, O₂, etc.), but which are of low energy (few eV). These excited species will bombard the surface of the supports and will therefore break some of the chemical bonds constituting this object (—C—C—, —C—O, —C—H, etc. bonds), which will give rise to “free-radical” species (—C^{o}; —O^{o}, etc.) which are generally unstable. It is to be estimated that the surface density of reactive species is from 10 to 100 radicals per square nanometer. Since the energy of the bombardment is low, the sites are created in a section of material (the support) less than 10 nanometers thick.

Without being bound by an interpretation, it may be envisioned that the radicals (unstable) will have several options in terms of what happens to them:

1) the radicals recombine with one another and the effect is lost,
2) the radicals recombine with antioxidants present in the material and the effect is lost,
3) the radicals recombine with oxygen and water atoms that are either in the material, but especially when placed in air again, and the effect is positive: the radicals are converted to stable chemical sites (such as —C—OH; —C═O, —C═O OH, etc.). These groups have a hydrophilic nature and provide the possibility of adhesion with something that will be provided: a paint, an adhesive, biocompatible polymers. The adhesion will be acquired spontaneously, through the establishment of low-energy bonds called hydrogen bonds. If said bonds are numerous, the adhesion will be excellent,
4) the radicals make it possible to initiate a polymerization, provided that they are placed in the presence of molecules capable of polymerizing, in an oxygen-free medium, and in general by heating for several hours: polymer chains are attached to the support via strong bonds, of covalent type. This system is more laborious to implement but the adhesion is excellent,
5) if the plasma is produced in the presence of a fluorinated gas, in particular CF₄, which is neither toxic nor aggressive, fluorine atoms will bind to the free radicals. —CF, —CF₂ and —CF₃ groups will form, said groups being very hydrophobic and therefore non-adhesive. This property makes it possible in particular to produce antiadhesive supports which are very useful as sample storage or transfer receptacles.

In the context of the invention, the receptacles or supports made of plastic can be chosen from the following nonlimiting list:
polypropylene (PP);
polyester terephthalate (PET);
polycarbonate (PC);
polystyrene (PS).

The supports used are commercial supports which have the desired size characteristics for the analyses (dishes, flat supports, multiwell systems, beads, etc.). They are clean and do not contain any pollutants that may originate from the manufacture.

In the context of the invention, the plasma treatment of the plastic receptacles or supports can be chosen from the following nonlimiting list:

### Types of Plasma:

carbon dioxide (CO₂);
carbon tetrafluoride (CF₄);
nitrogen (N₂);
oxygen (O₂);
argon (Ar).

In the context of the invention, the treatment by polymer deposition on the plastic receptacles or supports can be carried out with a solution of a polymer from the following nonlimiting list:

Chemical Treatments with a Polymer:
polyvinylpyrrolidone (or PVP) K30 and K90;
polyvinyl alcohol (or PVA) 88 and 98% in H₂O;
carboxymethylcellulose (or CMC);
0.2% hydroxypropylmethylcellulose (or HPMC);
polyethylene glycol (or PEG) E 4000;
polyhydroxyethyl methacrylate (or PHEMA).

### EXAMPLE

Obtaining a functionalized support according to the invention:

the PC-PVP K30 support.

A polycarbonate PCR strip containing 8 wells, sold by the German company RoboScreen (reference No. 0501000102), is used.

The plasma treatment of the polycarbonate support is carried out in a reactor with a volume of 20 liters operating under radio frequency. The reactor is placed under a vacuum in order to degas the support for 10 minutes. Nitrogen (N₂) is then introduced into the reactor at a flow rate of 10 cm³/min. For 10 minutes, the support is subjected to a working pressure of approximately 3.4×10⁻¹ mbar and a radioelectric power of 100 W. The support is then returned to atmospheric pressure, under a neutral atmosphere (N₂). Once returned to atmospheric pressure, the support is placed (for approximately 5 minutes and at ambient temperature) in a dilute aqueous solution of polyvinyl-pyrrolidone grade K30. The support is then completely dried, under a stream of air at approximately 40° C. Once dried, the support is stored under a neutral atmosphere (N₂) and in the dark in leaktight packages.

Other supports can be obtained in a similar manner, by optionally modifying the type of reactor used (radio-frequency or microwaves), the type of plasma used as indicated above, and the nature of the polymer used as indicated above.

The following supports were therefore produced for the studies:

Treatment with plasma alone:
PS (—N₂, —CO₂ and —CF₄)
PC (—N₂, —CO₂ and —CF₄)
PP (—N₂, —CO₂, —CF₄, Ar and O₂)

Treatment with plasma+polymer deposition:
PP-N₂ (-PVP, -PVA, -CMC, -HPMC, PEG and PHEMA)
PC-N₂ (-PVP, -PVA, -CMC, -HPMC, PEG and PHEMA)

### I) Analysis of the Supports Produced:

Physicochemical Analysis

The commercial supports used consist of:

95 to 100% of polymers (or plastics) such as poly-carbonate, polyester terephthalate, polystyrene or polypropylene,
0 to 5% of additives (liquid petroleum jelly, liquid paraffin or stearate).

These additives have the disadvantage of seeping out of the support as the days pass and of forming a polluting film covering the plastic surface.

Wetting Test

This test is carried out by means of the water drop test. During said test, a drop of water is deposited on the surface of the support and the internal angle of said drop is measured in order to estimate the hydrophobic (or hydrophilic) nature of the support.

### Nontreated Support

Contact angle of 90 to 95°, therefore relatively hydrophobic.

### Plasma-Treated Support

Contact angle of 20 to 30°, therefore relatively hydrophilic and generally relatively unstable over time.

In the specific case of fluorinated deposits:

Contact angle of 110 to 120°, therefore very hydro-phobic and generally very stable over time.

Support treated with plasma followed by polymer deposition

Contact angle of 20 to 40°, therefore relatively hydrophilic and generally very stable over time (several months).

### Use of the Supports for Immunodetection

### Immunodetection of the Recombinant Prion Protein:

### Solutions Used and Origins:

Coating buffer: 0.05M NaHCO₃−0.05M Na₂CO₃−pH 9.4.
PBS buffer: 0.14M NaCl−8 mM Na₂HPO₄.2H₂O−1.5 mM KH₂PO₄−2.7 mM KCl, pH 7.4.
Saturation buffer: PBS buffer+3% BSA−pH 7.4.
Dilution buffer A: PBS buffer+1% BSA+0.1% Tween 20−pH 7.4.
Dilution buffer B: PBS buffer+1% BSA−pH 7.4.
BSA washing buffer: PBS buffer+1.5% BSA−pH 7.4.
Tween washing buffer: PBS buffer+0.1% Tween 20−pH 7.4.
Monoclonal capture antibodies: SAF32 (Eurogentec) and 12F10 (Bio-Rad).
Monoclonal detection antibodies 12F10 coupled to peroxidase (Bio-Rad) and biotinylated 4F7 (Roboscreen).
Recombinant prion protein (PrPrec): origin Roboscreen and Prionics.
Streptavidin coupled to horseradish peroxidase (Strep-HRP): origin Dako.
3,3′,5,5′-tetramethylbenzedine (TMB): origin BD-Pharmingen.
Streptavidin coupled to a reporter DNA (Strep-DNA): origin Centre de Recherche sur les Protéines Prions [Center for Research on Prion Proteins] (Ulg)
PCR mix: 38% H₂O−12% primers−50% SYBRGreen

### Protocol “ELISA 1” (Antibody Couple: SAF32/12F10).

An ELISA plate containing 96 wells, sold by the German company Greiner (catalogue No. 655081) is used for this ELISA. The working volume is 50 μl/well.

The steps of the protocol are as follows:

1—Coating, overnight at 4° C., of the monoclonal capture antibody SAF32 diluted to a concentration of 1 μg/ml in coating buffer (50 μl/well).
2—Washing with 300 μl/well of PBS buffer (4 washes).
3—Blocking, for 2 hours at ambient temperature, of the sites not saturated with the capture antibody, with saturation buffer (300 μl/well).
4—Washing with 300 μl/well of PBS buffer (4 washes).
5—Incubation, for 1 hour at ambient temperature, of the bovine PrPrec protein (Roboscreen) diluted to various concentrations in dilution buffer A (50 μl/well).
6—Washes (300 μl/well): 2 in PBS buffer followed by 2 in BSA washing buffer.
7—Incubation, for 1 hour at ambient temperature, of the monoclonal detection antibody 12F10 coupled to peroxidase, diluted 10× in dilution buffer A (50 μl/well).
8—Washes (300 μl/well): 2 in Tween washing buffer followed by 2 in BSA washing buffer.
9—Incubation, for 30 minutes at ambient temperature and in the dark, of the peroxidase substrate, TMB (50 μl/well).
10—Stopping of the enzymatic reaction by adding 2N H₂SO₄ (25 μl/well).
11—Reading of the absorbance of the solution at 450 nm on a spectrophotometer (Labsystems Multiskans MS).
Protocol “ELISA 2” (Antibody Couple: 12F10/4F7 biot)

A strip containing 8 wells of ELISA type, sold by the German company Greiner (catalogue No. 762061), is used for this ELISA. The working volume is 100 μl/well.

The steps of the protocol are as follows:

1—Coating, overnight at 4° C., of the monoclonal capture antibody 12F10 diluted to a concentration of 10 μg/ml in coating buffer (100 μl/well).
2—Washing with 300 μl/well of PBS buffer (5 washes).
3—Blocking, for 2 hours at ambient temperature, of the sites not saturated with the capture antibody, with saturation buffer (300 μl/well).
4—Washing with 300 μl/well of PBS buffer (5 washes).
5—Incubation, for 1 hour at ambient temperature, of the bovine PrPrec protein (Prionics) diluted to various concentrations in dilution buffer B (100 μl/well).
6—Washing with 300 μl/well of PBS buffer (5 washes).
7—Incubation, for 1 hour at ambient temperature, of the biotinylated monoclonal detection antibody 4F7 diluted to 1 μg/ml in dilution buffer B (100 μl/well).
8—Washes (300 μl/well): 3 in Tween washing buffer followed by 3 in BSA washing buffer.
9—Incubation, for 30 minutes at ambient temperature, of the Strep-HRP diluted to 83 μg/ml in dilution buffer B (100 μl/well).
10—Washing with 300 μl/well of PBS buffer (5 washes).
12—Incubation, for 30 minutes at ambient temperature and in the dark, of the peroxidase substrate, TMB (100 μl/well).
13—Stopping of the enzymatic reaction by adding 2N H₂SO₄ (50 μl/well)
14—Reading of the absorbance of the solution at 450 nm on a spectrophotometer (Labsystems Multiskans MS).

Results of ELISA assays carried out on new supports prepared as described above.

I) Tests of PP Supports Treated with Plasma Alone (ELISA 1)

The performance level of the tubes was tested, in triplicate, according to the protocol ELISA 1 described above.

Optical density
(the value of the control without antigen already
deducted)

| PrP (ng/m) | PP control | PP CF₄ | PP CO₂ | PP N₂ |
|---|---|---|---|---|
| 1000 | 1.047 | 0.022 | 1.205 | 1.291 |
| 100 | 0.728 | 0.002 | 0.774 | 0.993 |
| 50 | 0.317 | 0.000 | 0.352 | 0.696 |
| 25 | 0.114 | 0.002 | 0.219 | 0.266 |
| 12.5 | 0.045 | 0.001 | 0.041 | 0.088 |
| 6.25 | 0.038 | 0.003 | 0.001 | 0.020 |
| 3.125 | 0.036 | 0.000 | 0.004 | 0.023 |

Detection limit (ng/ml)

| | | |
|---|---|---|
| 26.92 | 47.86 | 21.38 |

Detection range

| | | | |
|---|---|---|---|
| O.D. | ~0.9 | ~1 | ~1.1 |

| | | | |
|---|---|---|---|
| ng/ml | ~30 to 500 | ~50 to 1000 | ~20 to 1000 |

Discrimination power

| | | |
|---|---|---|
| ~0.52 | ~0.95 | ~0.89 |

Mean value of the background noise = 0.1

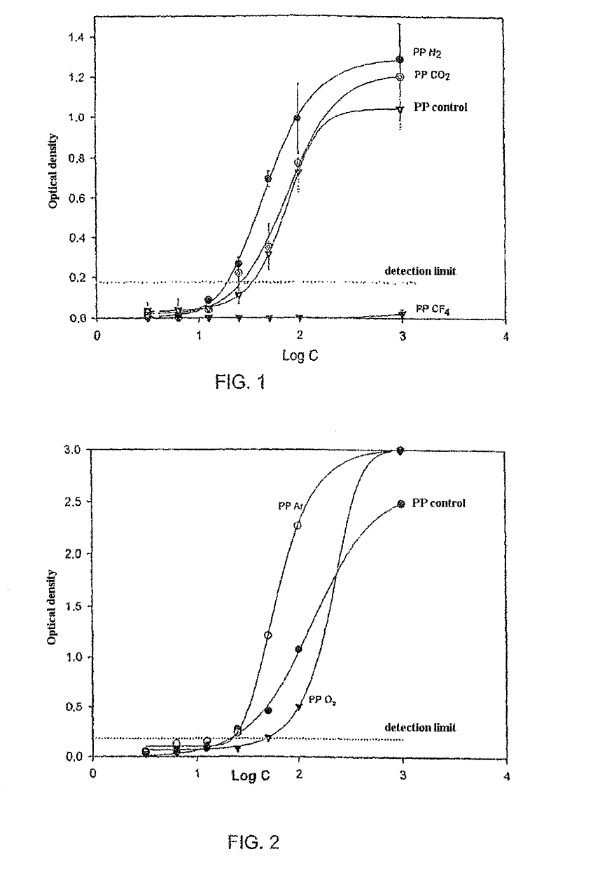

The results are also given in the form of a graph in FIG. 1.

These results indicate that the treatment with CF₄ plasma is not appropriate for assaying the molecule of interest, but could prove to be a judicious choice for the storage of molecules. For the immunodetection, the treatment with N₂ plasma gives the best results of this series.

II) Tests of the PP Supports Treated with Plasma Alone (ELISA 2)

The performance level of the tubes was tested, in duplicate, according to the protocol ELISA 2 described above.

Optical density
(the value of the control without antigen already
deducted)

| PrP (ng/m) | PP control | PP Ar | PP O₂ |
|---|---|---|---|
| 1000 | 2.488 | 3 | 3 |
| 100 | 1.076 | 2.271 | 0.505 |
| 50 | 0.46 | 1.212 | 0.19 |
| 25 | 0.276 | 0.237 | 0.083 |
| 12.5 | 0.086 | 0.153 | 0.124 |
| 6.25 | 0.045 | 0.132 | 0.073 |
| 3.125 | 0.052 | 0.037 | 0.033 |

Detection limit (ng/ml)

| | | |
|---|---|---|
| 22.91 | 15.85 | 67.61 |

Detection range

| | | | |
|---|---|---|---|
| O.D. | ~2.3 | ~2.8 | ~2.8 |
| ng/ml | ~20 to 1000 | ~15 to 1000 | ~70 to 1000 |

Discrimination power (ng/ml)

| | | |
|---|---|---|
| ~0.42 | ~0.35 | ~0.33 |

Mean value of the background noise = 0.11

The results are also given in the form of a graph in FIG. 2.

For this ELISA assay, the support modified with O₂ plasma is less effective than that modified with argon. An improvement in the detection limit by a factor close to 4 is observed for the support treated with argon, compared with that treated with O₂. Furthermore, the PP Ar has a greater detection range than the nontreated control, resulting in a better discrimination power. The latter therefore appears to be more suitable for this type of application.

III) Tests of PP Supports Treated with N₂ Plasma and Polymer Deposition

The performance level of the tubes was tested according to the protocol ELISA 2 described above.

Optical density
(the value of the control without antigen already deducted)

| PrP | PP | | | | |
|---|---|---|---|---|---|
| (ng/m) | control | PVP | PVA98 | CMC | HPMC |
| 4 | 0.021 | 0.257 | 0.19 | 0.116 | 0.173 |
| 20 | 0.087 | 1.66 | 1.138 | 1.119 | 1.176 |
| 100 | 0.824 | 3 | 2.747 | 2.826 | 3 |
| 1000 | 2.011 | 3 | 3 | 3 | 3 |

Detection limit (ng/ml)

| | | | | |
|---|---|---|---|---|
| 33.88 | 3.89 | 5.25 | 6.61 | 7.74 |

Detection range

| | | | | | |
|---|---|---|---|---|---|
| O.D. | ~1.8 | ~2.8 | ~2.8 | ~2.8 | ~2.8 |
| ng/ml | ~35 to 1000 | ~4 to 560 | ~5 to 560 | ~7 to 560 | ~8 to 560 |

Discrimination power (ng/ml)

| | | | | |
|---|---|---|---|---|
| ~0.54 | ~0.20 | ~0.20 | ~0.20 | ~0.20 |

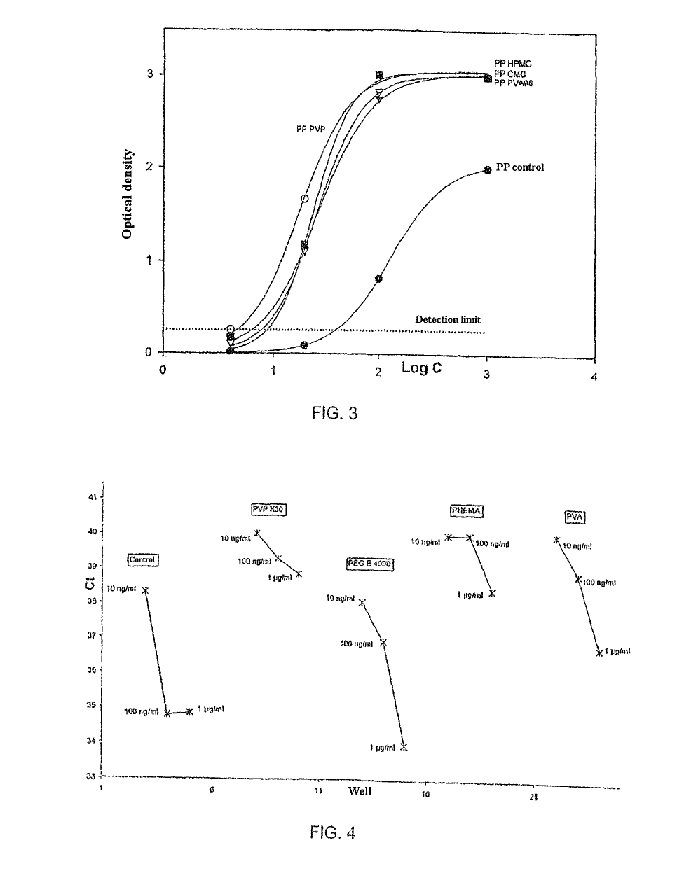

The results are also given in the form of a graph in FIG. 3.

All these polymer depositions, according to the invention, improve the detection limit by at least a factor of 4 and the discrimination power by at least a factor of 2.5.

Results of iPCRq assays carried out on new supports prepared as described above.

Protocol “iPCRq” (Antibody Couple: Saf32/4F7biot)

An iPCRq strip (Roboscreen) containing 8 wells is used for the iPCRq, and the working volume is 50 μl/well.

The steps of the protocol are as follows:

1—Coating, overnight at 4° C., of the monoclonal capture antibody Saf32 diluted to a concentration of 10 μg/ml in coating buffer (50 μl/well).
2—Washing with 250 μl/well of PBS buffer (5 washes).
3—Blocking, for 1 hour at 37° C., of the sites not saturated with the capture antibody, with saturation buffer (250 μl/well).
4—Washing with 250 μ/well of PBS buffer (5 washes).
5—Incubation, for 1 hour at ambient temperature, of the human PrPrec protein (Roboscreen) diluted to various concentrations in dilution buffer B (50 μ/well).
6—Washes (250 μl/well): 3 in Tween washing buffer followed by 3 in BSA washing buffer.
7—Incubation, for 1 hour at ambient temperature, of the biotinylated monoclonal detection antibody 4F7 diluted to 1 μg/ml in dilution buffer B (501/well).
8—Washes (250 μl/well): 3 in Tween washing buffer followed by 3 in BSA washing buffer.
9—Incubation, for 30 minutes at ambient temperature, of the Strep-DNA complex diluted in dilution buffer B (50 μl/well).
10—Washes (250 μl/well): 10 in PBS buffer followed by 10 in H₂O.
12—Addition of the PCR mix (50 μl/well).
13—Performing of PCR and reading of fluorescence in real time on an Applied Biosystems Gene Amp 5700 thermocycler:
10 minutes at 95° C. (activation of the Taq polymerase)
10 to 70 cycles of:
   15 seconds at 95° C. (denaturation)
   1 minute at 60° C. (oligonucleotides hybridization and extension).
   I) Tests of PC Supports Treated with N₂ Plasma and Polymer Deposition

The performance level of the tubes was tested according to the protocol iPCRq described above.

Threshold cycle (Ct)

| PrP | PC | | PEG E | | |
|---|---|---|---|---|---|
| (ng/m) | control | PVP K30 | 4000 | PHEMA | PVA |
| Without | 38.83 | 40.00 | 40.00 | 40.00 | 40.00 |
| Ag | | | | | |
| 10 | 38.31 | 40.00 | 38.06 | 40.00 | 40.00 |
| 100 | 34.79 | 39.28 | 36.93 | 38.43 | 38.92 |
| 1000 | 34.85 | 38.85 | 33.98 | 40.00 | 36.81 |

Detection limit (ng/ml)

| | | | | |
|---|---|---|---|---|
| <10 | 10>..<100 | <10 | 100>.<1000 | 10>.<100 |

The results are also given in the form of a graph in FIG. 4.

All these polymer depositions improve the detection threshold and make it possible to obtain a very low background noise for the control without antigen (Ct=40). One polymer deposition, PEG E 4000, has a better detection sensitivity range and is therefore found to be the best candidate, of this series, for carrying out tests for detecting the prion protein.

In general, the invention therefore provides
a transfer, storage or detection receptacle for solubilized or suspended entities (in particular biological entities), characterized in that it consists of a material (plastic, glass, mineral or organic compound), preferably made of polypropylene or polycarbonate, surface-treated with a plasma followed by polymer deposition;
the polymer deposition is obtained by soaking in or bringing into contact with a solution, preferably an aqueous solution, of an organic polymer, for example a polyethylene glycol, preferably at ambient temperature;
the polymer deposition is carried out directly after the plasma treatment, for example 1 to 10 minutes, preferably within 5 minutes;
the same receptacle can constitute a support for the assaying of biological entities, making it possible to minimize the losses of these molecules due to adsorption and/or due to impairments to their natural properties;
the same receptacle can also be a support for the transfer and handling of biological molecules or the like, reducing the losses of material on the walls thereof;
it may be isolated tubes, in the form of a multiwell strip or plate, or consumables of different shapes (tips, canulas, cupules);
the plasma used is a gas plasma (preferably N₂, Ar or CF₄), of varying pressure, power and frequency;
the treatment with plasma is followed by one or more depositions of organic molecules or of polymers of medical grade.

1. A method for treating a transfer, storage or detection receptacle for solubilized microbiological entities, in which the receptacle is surface-treated with an electromagnetic plasma followed by a polymer deposition procedure, at least on the walls intended to be in contact with the solution of microbiological entities, by soaking in a solution of said polymer.

2. The method as claimed in claim 1, for which the solution of polymer is an aqueous solution containing 0.05%-10% by weight, preferably 0.1% to 5%.

3. The method as claimed in claim 1, in which the procedure for soaking or bringing into contact with the solution of polymer is carried out within 10 minutes, preferably within 1 to 5 minutes, of the treatment with electromagnetic plasma.

4. The method as claimed in claim 1, characterized in that the plasma is an N₂, Ar or CR₄ gas plasma.

5. The method as claimed in claim 1, in which the polymer is polyvinylpyrrolidone, poly(vinyl alcohol), cellulose derivatives (CMC or HPMC), polyethylene glycol (PEG) or polyhydroxyethyl methacrylate (PHEMA).

6. The method as claimed in claim 1, characterized in that the polymer is polyethylene glycol PEG E 4000.

7. A receptacle obtained by means of a method as claimed in claim 1.

8. The receptacle as claimed in claim 7, characterized in that it is an isolated tube, tubes in the form of a multiwell strip or plate, or a consumable of varied shapes.

9. The receptacle as claimed in claim 8, characterized in that it is made of polypropylene, polycarbonate or polystyrene.

10-14. (canceled)

15. A method for detecting and/or assaying a microbiological entity, characterized in that an ELISA, i-PCR or ligand-PCR technique is used and that at least one receptacle of solutions to be analyzed is a receptacle as claimed in claim 1.

16. The method as claimed in claim 15, in which the deposited or grafted polymer is polyethylene glycol, for example PEG E 4000.

17. The method as claimed in claim 15, in which the microbiological entity is a prion protein.

18. A kit for assaying microbiological entities, comprising at least one receptacle or support as claimed in claims 7.

19. The kit as claimed in claim 18, also comprising at least one container for storing or transferring a solution of said microbiological entities, or receptacle, and whose walls in contact with said solution have been treated with a CF₄ plasma.
